(19) United States
(12) Patent Application Publication
(10) Pub. No.: US 2024/0122464 A1
(43) Pub. Date: Apr. 18, 2024

(54) SINGLE-OPERATOR LAPAROSCOPE AUXILIARY OPERATING DEVICE

(71) Applicant: UNIVERSITY-TOWN HOSPITAL OF CHONGQING MEDICAL UNIVERSITY, Chongging (CN)

(72) Inventors: Bo TANG, Yuzhong District, Chongqing (CN); Hailong LUO, Yuzhong District, Chongqing (CN); Haolong ZHANG, Yuzhong District, Chongqing (CN); Kun RAN, Yuzhong District, Chongqing (CN)

(21) Appl. No.: 18/251,112

(22) PCT Filed: Feb. 2, 2023
(86) PCT No.: PCT/CN2023/074218

§ 371 Date: Apr. 28, 2023

(30) Foreign Application Priority Data
Mar. 2, 2022 (CN) 202210199020.0

(51) Int. Cl. A61B 1/313; A61B 1/00; A61B 1/045

(52) U.S. Cl. CPC A61B 1/3132; A61B 1/00147; A61B 1/045

(57) ABSTRACT

A single-operator laparoscope auxiliary operating device is provided, including a laparoscope and universal rotating mechanisms, wherein two universal rotating mechanisms are provided on the laparoscope, each of the universal rotating mechanisms includes a ball sleeve and a sleeve plate, the ball sleeve is slidably sleeved on the laparoscope, the sleeve plate is sleeved in a middle of the ball sleeve, and the ball sleeve is universally rotatable on the sleeve plate; a direction controlling mechanism; a moving mechanism; a supporting mechanism; a connecting frame, one end of the connecting frame is fixedly connected to the sleeve plate close to the lens of the laparoscope, and another end of the connecting frame is fixedly connected to the supporting mechanism; and a foot controller connected to the direction controlling mechanism and the moving mechanism.

### CROSS REFERENCE TO RELATED APPLICATION

This application is a national stage application of International Patent Application No. PCT/CN2023/074218, filed on Feb. 2, 2023, which claims priority of the Chinese Patent Application No. 202210199020.0, filed on Mar. 2, 2022, both of which are incorporated by references in their entities.

### FIELD

The present disclosure relates to the technical field of laparoscope, and in particular, to a single-operator laparoscope auxiliary operating device.

### INTRODUCTION

At present, the laparoscopic minimally invasive surgery is a research hotspot in medical technology, and is a development trend of future surgery. This is mainly because the minimally invasive medical treatment has many advantages, such as small trauma, short hospital stay, fast recovery, and few post-operative complications. With the improvement of medical equipment and doctor's skill level, the laparoscopic surgery is increasingly used in a wide variety of surgeries. The laparoscopic surgery requires other medical devices to assist the work, and thereby requires an assistant to hold the laparoscope, and the position of the laparoscope is changed in real time based on the operation of the surgeon.

However, the assistant holding the laparoscope for a long time are prone to fatigue, resulting in tremor and paralysis of the hands. As a result, the fluency and stability of the surgical operation of the surgeon are affected, and the surgery risk is increased. Further, the assistant need to match the surgeon in a tacitly manner. If the assistant cannot understand the intention of the surgeon, the assistant not only affect the surgery progress, but also may cause accidental injury.

### SUMMARY

With regard to the described problems existing in the prior art, the technical problem to be solved by the present disclosure is: the assistant need to match the surgeon in a tacitly manner, if the assistant cannot understand the intention of the surgeon, the assistant not only affect the surgery progress, but also may cause accidental injury.

In order to solve the described technical problem, the present disclosure adopts the following technical solution: a single-operator laparoscope auxiliary operating device, comprising: a laparoscope; universal rotating mechanisms, wherein two universal rotating mechanisms are provided on the laparoscope, each of the universal rotating mechanisms comprises a ball sleeve and a sleeve plate, the ball sleeve are slidably sleeved on the laparoscope, the sleeve plate is sleeved in a middle of the ball sleeve, and the ball sleeve is universally rotatable on the sleeve plate; a direction controlling mechanism for controlling the sleeve plate away from a lens of the laparoscope to move in a plane; a moving mechanism for controlling the laparoscope to slide in the ball sleeve; a supporting mechanism for supporting the direction controlling mechanism; a connecting frame, one end of the connecting frame is fixedly connected to the sleeve plate close to the lens of the laparoscope, another end of the connecting frame is fixedly connected to the supporting mechanism; and a foot controller connected to the direction controlling mechanism and the moving mechanism, for controlling operations of the direction controlling mechanism and the moving mechanism.

In the present disclosure, the supporting mechanism is moved to adjust positions of the laparoscope and the laparoscope is inserted into abdominal cavity. After the laparoscope is mounted, when it is necessary to move positions of the lens of the laparoscope, the direction controlling mechanism is controlled to operate by the surgeon via the foot controller, the direction controlling mechanism drives the sleeve plate away from the lens of the laparoscope to move on the plane, so that the sleeve plate away from the lens of the laparoscope drives the laparoscope to rotate on the sleeve plate away from the lens of the laparoscope with the ball sleeve close to the lens of the laparoscope as a center, so as to drive the lens of the laparoscope to illuminate a specified area in the abdominal cavity; when the lens of the laparoscope is needed to move forwards or backwards, the moving mechanism is controlled to operate by the surgeon via the foot controller, the moving mechanism controls the lens of the laparoscope to slide in the ball sleeve, so as to obtain images from different distances via the lens of the laparoscope. By replacing the work of the assistant with the single-operator laparoscope auxiliary operating device, the tremor and paralysis of the hands of the assistant caused by holding the laparoscope for a long-time and thereby the increased surgery risk can be effectively avoided; further, the surgeon can control the movement of the lens of the laparoscope by his/her intention to obtain required surgery images, which can accelerate the surgery progress, and can also effectively prevent mutual interference between laparoscope and equipment of the surgeon, reducing the risk of accidental injure.

Preferably, the foot controller is detachably mounted on the supporting mechanism, and when the foot controller is needed to be used, the foot controller can be removed from the supporting mechanism for operation.

Preferably, the direction controlling mechanism comprises a rectangular rail, four sliding sleeves, a first telescopic member, a second telescopic member, a third telescopic member and a fourth telescopic member, the rectangular rail is fixedly mounted on the supporting mechanism, and the rectangular rail is formed by four rail edges; the four sliding sleeves are slidably mounted on each of the rail edges of the rectangular rail respectively; the first telescopic member, the second telescopic member, the third telescopic member and the fourth telescopic member are in correspondence with the four sliding sleeves one by one; one end of each of the first telescopic member, the second telescopic member, the third telescopic member and the fourth telescopic member is fixedly connected to a corresponding sliding sleeve, and another end of each of the first telescopic member, the second telescopic member, the third telescopic member and the fourth telescopic member is fixedly connected to the sleeve plate away from the lens of the laparoscope. By means of the telescoping of the first telescopic member, the second telescopic member, the third telescopic member and the fourth telescopic member, the sleeve plate away from the lens of the laparoscope can be controlled to move in the plane, so that the positions of the lens of laparoscope in the abdominal cavity can be adjusted.

Preferably, the moving mechanism comprises a fixed sleeve, a motor, a threaded rod and a pushing structure. The fixed sleeve is sleeved on one end of the laparoscope away from the lens of the laparoscope, and the fixed sleeve is fixedly connected to one end of the ball sleeve away from the lens of the laparoscope; the motor is mounted in the fixed sleeve, the motor drives the threaded rod to rotate, and the foot controller controls the motor to rotate forwards and reversely; the pushing structure is slidably connected in the fixed sleeve, the pushing structure is detachably connected to the laparoscope, and the pushing structure is in threaded connection with the threaded rod. The motor is operated to drive the threaded rod to rotate, the threaded rod drives the pushing structure in threaded connection therewith to slide, and the pushing structure pushes the laparoscope to move in the ball sleeve along a linear direction of the laparoscope, thereby adjusting depths of the lens of the laparoscope in the abdominal cavity, so as to obtain surgery images from different distances.

Preferably, the supporting mechanism comprises a base, a height adjusting member, a length adjusting member and a fixing frame. The base is fixedly connected to one end of the height adjusting member, and another end of the height adjusting member is vertically fixedly connected to one end of the length adjusting member; another end of the length adjusting member is hinged to the fixing frame; the fixing frame is fixedly connected to the direction controlling mechanism, and the fixing frame is fixedly connected to one end of the connecting frame away from the sleeve plate; universal wheels are also mounted at a bottom of the base. The general positions of the laparoscope are adjusted by the height adjusting member and the length adjusting member, and the general angles of the laparoscope are adjusted by a joint between the length adjusting member and the fixing frame, so that the laparoscope can be inserted into the abdominal cavity more easily, and the single-operator laparoscope auxiliary operating device can be moved by means of the universal wheels.

Preferably, a self-locking mechanism is further comprised, the self-locking mechanism is mounted on the ball sleeve close to the lens of the laparoscope, when the direction controlling mechanism operates, the self-locking mechanism locks the laparoscope and the ball sleeve close to the lens of the laparoscope; when the moving mechanism operates, the self-locking mechanism releases the laparoscope and the ball sleeve close to the lens of the laparoscope, wherein the self-locking mechanism is provided with a pan-tilt. The self-locking mechanism can effectively prevent the laparoscope from sliding in the ball sleeve when the direction controlling mechanism operates, thereby increasing accuracy of the movement of the lens of the laparoscope, furthermore, the self-locking mechanism is provided with the pan-tilt, which can achieves the anti-shaking function, and prevent the lens of the laparoscope from shaking.

Preferably, the pushing structure comprises a moving block, a friction column, a reset spring, a pushing rod, a rotating plate, a cam, and a limiting rod. The moving block is slidably connected in the fixed sleeve, and the moving block is in threaded connection with the threaded rod; the friction column is located in the moving block and abuts against the threaded rod; one end of the reset spring is fixedly connected to the friction column, and another end of the reset spring is fixedly connected to the rotating plate; the pushing rod is slidably provided in the moving block, one end of the pushing rod is provided with a rotating groove, and the rotating plate is rotatably connected in the rotating groove; another end of the pushing rod is detachably connected to the laparoscope; the cam is located in the rotating groove and is coaxially fixed on the rotating plate; one end of the limiting rod is fitted with the cam, and another end of the limiting rod is slidably inserted through the rotating groove and is frictionally connected to the moving block.

The threaded rod continuously rotates, so that the friction column can be driven to rotate by the friction force, the friction column drives the reset spring to rotate, and the reset spring drives the cam to rotate via the rotating plate, the cam pushes the limiting rod to closely fit with the moving block, so as to clamp the pushing rod and the moving block, so that the movement of the moving block can drive the pushing rod to move, and the pushing rod pushes the laparoscope to move; when the threaded rod is stopped rotating, since the reset spring has a certain reverse torque when rotating, the reverse torque will drive the cam to reversely rotate, so that the cam is separated from the limiting rod, and the pushing rod can slide again in the moving block; when the reverse torque is greater than the friction force between the threaded rod and the friction column, the relative sliding may occur between the threaded rod and the friction column, thereby effectively preventing the reset spring from being damaged as the excessive torque; when the direction controlling mechanism controls the sleeve plate away from the lens of the laparoscope to move on the plane, the laparoscope will slide in the ball sleeve away from the lens of the laparoscope and the fixed sleeve, the pushing rod slides in the moving block to eliminate the effect of the pushing force caused by the laparoscope.

Preferably, a protrusion edge and limiting mechanisms are further comprised. Each of the limiting mechanisms comprises arc rods and spiral springs; one end of the ball sleeve is provided with the protrusion edge; the sleeve plate is provided with arc groove; one end of each of the arc rods is slidably arranged in a corresponding arc groove; another end of each of the arc rods abuts against a corresponding protrusion edge; the arc rods are concentrically provided with the ball sleeve; each of the spiral springs is provided on a corresponding sleeve plate, a central end of each of the spiral springs is fixedly connected to the sleeve plate, and an outer end of each of the spiral springs is fixedly connected to a corresponding arc rod; each of the spiral springs is able to drive the corresponding arc rod to reset. The arc rods can limit the positions of the ball sleeve, which can effectively prevent the ball sleeve and the sleeve plate from falling off. When the direction controlling mechanism 3 operates, the ball sleeve rotates in the sleeve plate, and the ball sleeve drives the corresponding protrusion edge to press the corresponding arc rod into the corresponding arc groove, and at the same time, each of the arc rods drives the corresponding spiral spring to extend, so that the corresponding spiral spring stores energy, when the ball sleeve rotates reversely, the corresponding arc rod may be driven to protrude by the corresponding spiral spring.

Preferably, one end of each of the arc rods abutting against the corresponding protrusion edge is provided with a rolling ball for reducing friction force between each of the arc rods and the corresponding protrusion edge.

Preferably, the foot controller is provided with an up key, a down key, a left key, a right key, a zoom-in key and a zoom-out key. The up key and the down key are both connected to the first telescopic member and the second telescopic member; the left key and the right key are both connected to the third telescopic member and the fourth telescopic member; the zoom-in key and the zoom-out key are both connected to the motor. The foot controller is connected to the direction controlling mechanism and the moving mechanism via data lines; alternatively, the foot controller is wirelessly connected to the direction controlling mechanism and the moving mechanism. When the up key is pressed, the first telescopic member is extended and the second telescopic member is shorted; when the down key is pressed, the second telescopic member is extended and the first telescopic member is shorted; when the left key is pressed, the third telescopic member is extended and the fourth telescopic member is shorted; when the right key is pressed, the fourth telescopic member is extended and the third telescopic member is shorted; when the zoom-in key is pressed, the motor rotates forwards, and the laparoscope moves into the abdominal cavity; and when the zoom-out key is pressed, the motor rotates reversely, and the laparoscope moves out of the abdominal cavity.

Compared with the prior art, the present disclosure has at least the following advantages:
1. It can effectively avoid the laparoscope and equipment of the surgeon from interfering each other. In the present disclosure, the surgeon can control the movement of the lens of the laparoscope by his/her intention to obtain required surgery images, which can accelerate the surgery progress, and can also effectively prevent mutual interference between laparoscope and equipment of the surgeon, reducing the risk of accidental injure.
2. The work of the assistant can be effectively replaced, thereby reducing labor costs. In the present disclosure, the direction controlling mechanism is controlled to operate via the foot controller, the direction controlling mechanism drives the sleeve plate away from the lens of the laparoscope to move in the plane, so that the sleeve plate away from the lens of the laparoscope drives the laparoscope to rotate on the sleeve plate away from the lens of the laparoscope with the ball sleeve close to the lens of the laparoscope as the center, so as to drive the lens of the laparoscope to illuminate a specified area in the abdominal cavity; when the lens of the laparoscope is needed to move forwards or backwards, the moving mechanism is controlled to operate by the surgeon via the foot controller, the moving mechanism controls the lens of the laparoscope to slide in the ball sleeve, so as to obtain images from different distances via the lens of the laparoscope. In this way, the work of the assistant are replaced.

### BRIEF DESCRIPTION OF THE DRAWINGS

To illustrate the embodiments of the present disclosure more clearly, the accompanying drawings required for describing the embodiments are introduced briefly in the following. In all of the accompanying drawings, elements or portions are not necessarily drawn to the actual scale.

FIG. 1 is an overall schematic diagram of a single-operator laparoscope auxiliary operating device provided by an embodiment of the present disclosure.

FIG. 2 is a perspective view of the single-operator laparoscope auxiliary operating device provided by an embodiment of the present disclosure.

FIG. 3 is a perspective view of a universal rotating mechanism provided by an embodiment of the present disclosure.

FIG. 4 is a partial perspective view of structures at a laparoscope provided by an embodiment of the present disclosure.

FIG. 5 is a sectional view of a moving mechanism provided by an embodiment of the present disclosure.

FIG. 6 is a structural diagram of structures at a pushing structure provided by an embodiment of the present disclosure.

FIG. 7 is a structural diagram of a self-locking mechanism provided by an embodiment of the present disclosure.

FIG. 8 is a structural diagram of a limiting mechanism provided by an embodiment of the present disclosure.

FIG. 9 is a structural diagram of a foot controller provided by an embodiment of the present disclosure.

Reference numerals: 1—laparoscope, 2—universal rotating mechanism, 21—ball sleeve, 22—sleeve plate, 23—protrusion edge, 24—limiting mechanism, 241—arc rod, 242—spiral spring, 243—arc groove, 244—rolling ball, 3—direction controlling mechanism, 31—rectangular rail, 32—sliding sleeve, 33—first telescopic member, 34—second telescopic member, 35—third telescopic member, 36—fourth telescopic member, 4—moving mechanism, 41—fixed sleeve, 42—motor, 43—threaded rod, 44—pushing structure, 441—moving block, 442—friction column, 443—reset spring, 444—pushing rod, 445—rotating plate, 446—cam, 447—limiting rod, 448—rotating groove, 5—supporting mechanism, 51—base, 52—height adjusting member, 53—length adjusting member, 54—fixed frame, 6—connecting frame, 7—foot controller, 8—self-locking mechanism.

### DETAILED DESCRIPTION

The following embodiments of the present disclosure will be described in detail with reference to the accompanying drawings. The following embodiments are only used to illustrate the technical solutions of the present disclosure more clearly, and therefore are only used as examples and cannot be used to limit the scope of the present disclosure.

In addition, the terms “first”, “second”, and the like are only used for purposes of description, and cannot be understood as indicating or implying relative importance or implicitly indicating the number of indicated technical features. In the description of the present disclosure, “a plurality of” means two or more unless specified otherwise.

In the present disclosure, unless specified or limited otherwise, the terms “mount”, “join”, “connect”, “fix” and the like are used broadly, and may be, for example, fixed connections, detachable connections, or integral connections; may also be mechanical or electrical connections; may also be direct connections or indirect connections via intervening structures; may also be inner communications of two elements or interaction relationships between two elements. The specific meanings of the above terms in the present disclosure can be understood by those skilled in the art according to specific situations.

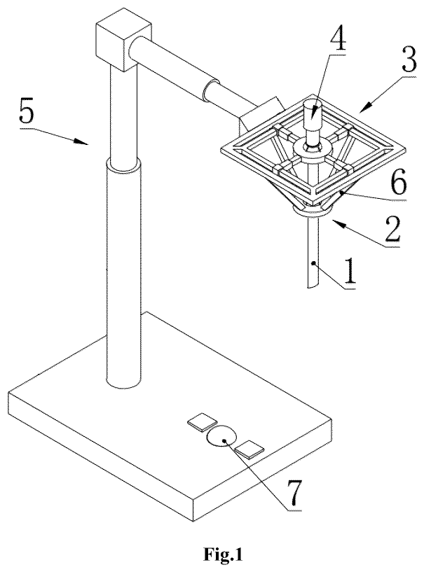

Referring to FIGS. 1-3, the present disclosure provides an embodiment of a single-operator laparoscope auxiliary operating device, including: a laparoscope 1; universal rotating mechanisms 2, wherein two universal rotating mechanisms 2 are provided on the laparoscope 1, each of the universal rotating mechanisms 2 includes a ball sleeve 21 and a sleeve plate 22, the ball sleeve 21 is slidably sleeved on the laparoscope 1, the sleeve plate 22 is sleeved in a middle of the ball sleeve 21, and the ball sleeve 21 is universally rotatable on the sleeve plate 22; a direction controlling mechanism 3 for controlling the sleeve plate 22 away from a lens of the laparoscope 1 to move in a plane; a moving mechanism 4 for controlling the laparoscope 1 to slide in the ball sleeves 21; a supporting mechanism 5 for supporting the direction controlling mechanism 3; a connecting frame 6, one end of the connecting frame 6 is fixedly connected to the sleeve plate 22 close to the lens of the laparoscope 1, another end of the connecting frame 6 is fixedly connected to the supporting mechanism 5; and a foot controller 7 connected to the direction controlling mechanism 3 and the moving mechanism 4, for controlling operations of the direction controlling mechanism 3 and the moving mechanism 4.

During specific implementation, the supporting mechanism 5 is moved to adjust the positions of the laparoscope land the laparoscope 1 is inserted into the abdominal cavity. After installing the laparoscope 1, when it is necessary to move the positions of the lens of the laparoscope 1, the direction controlling mechanism 3 is controlled to operate by the surgeon via the foot controller 7, the direction controlling mechanism 3 drives the sleeve plate 22 away from the lens of the laparoscope 1 to move on the plane, so that the sleeve plate 22 away from the lens of the laparoscope 1 drives the laparoscope 1 to rotate on the sleeve plate 22 away from the lens of the laparoscope 1 with the ball sleeve 21 close to the lens of the laparoscope 1 as the center, so as to drive the lens of the laparoscope 1 to illuminate a specified area in the abdominal cavity; when the lens of the laparoscope 1 is needed to move forwards or backwards, the moving mechanism 4 is controlled to operate by the surgeon via the foot controller 7, the moving mechanism 4 controls the lens of the laparoscope 1 to slide in the ball sleeves 21, so as to obtain images from different distances via the lens of the laparoscope 1. By replacing the work of the assistant with the single-operator laparoscope auxiliary operating device, the tremor and paralysis of the hands of the assistant caused by holding the laparoscope for a long-time and thereby the increased surgery risk can be effectively avoided; further, the surgeon can control the movement of the lens of the laparoscope 1 by his/her intention to obtain required surgery images, which can accelerate the surgery progress, and can also effectively prevent mutual interference between laparoscope 1 and equipment of the surgeon, reducing the risk of accidental injure.

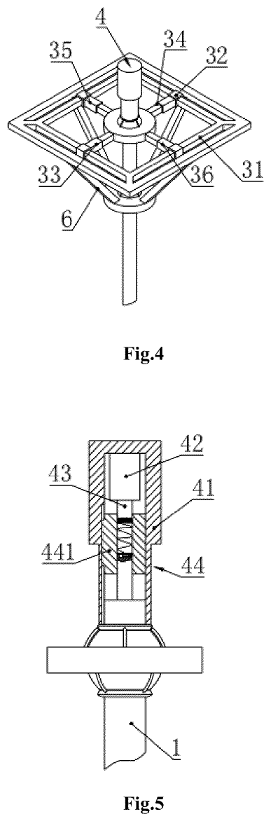

Referring to FIG. 4, in other embodiments, the direction controlling mechanism 3 may be of the following structures, including a rectangular rail 31, four sliding sleeves 32, a first telescopic member 33, a second telescopic member 34, a third telescopic member 35 and a fourth telescopic member 36. The rectangular rail 31 is fixedly mounted on the supporting mechanism 5, and the rectangular rail 31 is formed by four rail edges; the four sliding sleeves 32 are slidably mounted on each of the rail edges of the rectangular rail 31 respectively; the first telescopic member 33, the second 8 telescopic member 34, the third telescopic member 35 and the fourth telescopic member 36 are in correspondence with the four sliding sleeves 32 one by one; one end of each of the first telescopic member 33, the second telescopic member 34, the third telescopic member 35 and the fourth telescopic member 36 is fixedly connected to the sliding sleeve 32, and another end of each of the first telescopic member 33, the second telescopic member 34, the third telescopic member 35 and the fourth telescopic member 36 is fixedly connected to the sleeve plate 22 away from the lens of the laparoscope 1. By means of the telescoping of the first telescopic member 33, the second telescopic member 34, the third telescopic member 35 and the fourth telescopic member 36, the sleeve plate 22 away from the lens of the laparoscope 1 can be controlled to move on the plane, so that the positions of the lens of laparoscope 1 in the abdominal cavity can be adjusted.

Referring to FIG. 5, in other embodiments, the moving mechanism 4 may be of the following structures, including a fixed sleeve 41, a motor 42, a threaded rod 43 and a pushing structure 44. The fixed sleeve 41 is sleeved on one end of the laparoscope 1 away from the lens of the laparoscope 1, and the fixed sleeve 41 is fixedly connected to one end of the ball sleeve 21 away from the lens of the laparoscope 1; the motor 42 is mounted in the fixed sleeve 41, the motor 42 drives the threaded rod 43 to rotate, and the foot controller 7 controls the motor 42 to rotate forwards and reversely; the pushing structure 44 is slidably connected in the fixed sleeve 41, the pushing structure 44 is detachably connected to the laparoscope 1, and the pushing structure 44 is in threaded connection with the threaded rod 43. During specific implementation, the motor 42 is operated to drive the threaded rod 43 to rotate, the threaded rod 43 drives the pushing structure 44 in threaded connection therewith to slide, and the pushing structure 44 pushes the laparoscope 1 to move in the ball sleeve 21 along the linear direction of the laparoscope 1, thereby adjusting the depths of the lens of the laparoscope 1 in the abdominal cavity, so as to obtain surgery images from different distances.

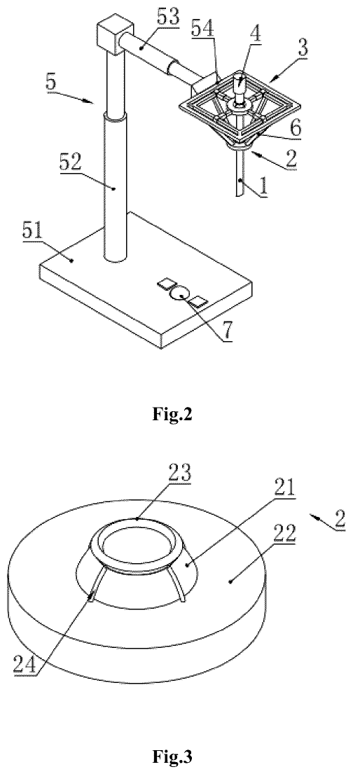

Referring to FIGS. 1 and 2, in other embodiments, the supporting mechanism 5 may be of the following structures, including a base 51, a height adjusting member 52, a length adjusting member 53 and a fixing frame 54. The base 51 is fixedly connected to one end of the height adjusting member 52, and another end of the height adjusting member 52 is vertically fixedly connected to one end of the length adjusting member 53; another end of the length adjusting member 53 is hinged to the fixing frame 54; the fixing frame 54 is fixedly connected to the direction controlling mechanism 3, and the fixing frame 54 is fixedly connected to one end of the connecting frame 6 away from the sleeve plate 22; universal wheels are also mounted at a bottom of the base 51. During specific implementation, the general positions of the laparoscope 1 are adjusted by the height adjusting member 52 and the length adjusting member 53, and the general angles of the laparoscope 1 are adjusted by the joint between the length adjusting member 53 and the fixing frame 54, so that the laparoscope 1 can be inserted into the abdominal cavity more easily, and the single-operator laparoscope auxiliary operating device can be moved by means of the universal wheels.

Furthermore, the base 51 is of a rectangular structure, and the universal wheels are mounted at four corners of the bottom of the base 51.

Referring to FIG. 7, in other embodiments, a self-locking mechanism 8 is further included, the self-locking mechanism 8 is mounted on the ball sleeve 21 close to the lens of the laparoscope 1, when the direction controlling mechanism 3 operates, the self-locking mechanism 8 locks the laparoscope 1 and the ball sleeve 21 close to the lens of the laparoscope 1; when the moving mechanism 4 operates, the self-locking mechanism 8 releases the laparoscope 1 and the ball sleeve 21 close to the lens of the laparoscope 1, wherein the self-locking mechanism 8 is provided with a pan-tilt. During specific implementation, the self-locking mechanism 8 includes a circular sleeve, a bottom block, a rotating rod, a friction block, a connecting rod and a fifth telescopic member; the circular sleeve is sleeved on the laparoscope 1, one end of the bottom block is fixedly connected to the circular sleeve, another end of the bottom block is hinged to a middle of the rotating rod, one end of the rotating rod is fixedly connected to the friction block, another end of the rotating rod is hinged to one end of the connecting rod, and another end of the connecting rod is hinged to the fifth telescopic member; the friction block is fitted with the laparoscope 1; and the friction block is driven to be fitted with and separated from the laparoscope 1 by the telescoping of the fifth telescopic member, so that the self-locking mechanism 8 is locked and released, and the self-locking mechanism 8 can effectively prevent the laparoscope 1 from sliding in the ball sleeve 21 when the direction controlling mechanism 3 operates, thereby increasing the accuracy of the movement of the lens of the laparoscope 1, furthermore, the self-locking mechanism 8 is provided with the pan-tilt, which can achieves the anti-shaking function, and prevent the lens of the laparoscope 1 from shaking.

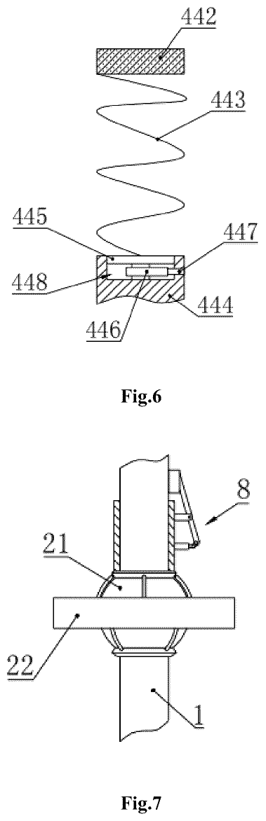

Referring to FIGS. 5 and 6, in other embodiments, the pushing structure 44 may be of the following structures, including a moving block 441, a friction column 442, a reset spring 443, a pushing rod 444, a rotating plate 445, a cam 446, and a limiting rod 447. The moving block 441 is slidably connected in the fixed sleeve 41, and the moving block 441 is in threaded connection with the threaded rod 43; the friction column 442 is located in the moving block 441 and abuts against the threaded rod 43; one end of the reset spring 443 is fixedly connected to the friction column 442, and another end of the reset spring 443 is fixedly connected to the rotating plate 445; the pushing rod 444 is slidably provided in the moving block 441, one end of the pushing rod 444 is provided with a rotating groove 448, and the rotating plate 445 is rotatably connected in the rotating groove 448; another end of the pushing rod 444 is detachably connected to the laparoscope 1; the cam 446 is located in the rotating groove 448 and is coaxially fixed on the rotating plate 445; one end of the limiting rod 447 is fitted with the cam 446, and another end of the limiting rod 447 is slidably inserted through the rotating groove 448 and is frictionally connected to the moving block 441.

During specific implementation, the threaded rod 43 continuously rotates, so that the friction column 442 can be driven to rotate by the friction force, the friction column 442 drives the reset spring 443 to rotate, and the reset spring 443 drives the cam 446 to rotate via the rotating plate 445, the cam 446 pushes the limiting rod 447 to closely fit with the moving block 441, so as to clamp the pushing rod 444 and the moving block 441, so that the movement of the moving block 441 can drive the pushing rod 444 to move, and the pushing rod 444 pushes the laparoscope 1 to move; when the threaded rod 43 is stopped rotating, since the reset spring 443 has a certain reverse torque when rotating, the reverse torque will drive the cam 446 to reversely rotate, so that the cam 446 is separated from the limiting rod 447, and the pushing rod 444 can slide again in the moving block 441; when the reverse torque is greater than the friction force between the threaded rod 43 and the friction column 442, the relative sliding may occur between the threaded rod 43 and the friction column 442, thereby effectively preventing the reset spring 443 from being damaged as the excessive torque; when the direction controlling mechanism 3 controls the sleeve plate 22 away from the lens of the laparoscope 1 to move on the plane, the laparoscope 1 will slide in the ball sleeve 21 away from the lens of the laparoscope land the fixed sleeve 41, the pushing rod 444 slides in the moving block 441 to eliminate the effect of the pushing force caused by the laparoscope 1. Specifically, in order to ensure that the laparoscope 1 can be pulled out by the pushing structure 44, a tension spring is provided between the moving block 441 and the laparoscope 1, and the laparoscope 1 can be pulled up by the tension spring.

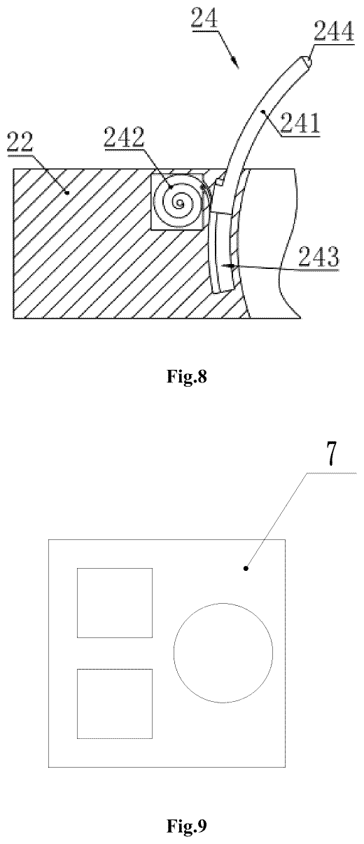

Referring to FIGS. 3 and 8, in other embodiments, protrusion edges 23 and limiting mechanisms 24 are further included. Each of the limiting mechanisms 24 includes arc rods 241 and spiral springs 242; one end of the ball sleeve 21 is provided with the protrusion edge 23; the sleeve plate 22 is provided with arc groove 243; one end of each of the arc rods 241 is slidably provided in a corresponding arc groove 243; another end of each of the arc rods 241 abuts against the corresponding protrusion edge 23; the arc rods 241 are concentrically provided with the ball sleeve 21; each of the spiral springs 242 is provided on the corresponding sleeve plate 22, a central end of each of the spiral springs 242 is fixedly connected to the sleeve plate 22, and an outer end of each of the spiral springs 242 is fixedly connected to the corresponding arc rod 241; each of the spiral springs 242 can drive the corresponding arc rod 241 to reset. Furthermore, in order to reduce the friction force between the arc rod 241 and the protrusion edge 23, one end of each of the arc rods 241 abutting against the protrusion edge 23 is provided with a rolling ball 244. During specific implementation, the arc rods 241 can limit the positions of the ball sleeve 21, which can effectively prevent the ball sleeve 21 and the sleeve plate 22 from falling off. When the direction controlling mechanism 3 operates, the ball sleeve 21 rotates on the sleeve plate 22, and the ball sleeve 21 drives the protrusion edge 23 to press the arc rod 241 into the arc grooves 243, and at the same time, the arc rod 241 drives the spiral spring 242 to extend, so that the spiral spring 242 stores energy, when the ball sleeve 21 rotates reversely, the arc rod 241 may be driven to protrude by the spiral spring 242.

In another embodiment, the foot controller 7 is provided with an up key, a down key, a left key, a right key, an zoom-in key and a zoom-out key. The up key and the down key are both connected to the first telescopic member 33 and the second telescopic member 34; the left key and the right key are both connected to the third telescopic member 35 and the fourth telescopic member 36; the zoom-in key and the zoom-out key are both connected to the motor 42. During specific implementation, when the up key is pressed, the first telescopic member 33 is extended and the second telescopic member 34 is shorted; when the down key is pressed, the second telescopic member 34 is extended and the first telescopic member 33 is shorted; when the left key is pressed, the third telescopic member 35 is extended and the fourth telescopic member 36 is shorted; when the right key is pressed, the fourth telescopic member 36 is extended and the third telescopic member 35 is shorted; when the zoom-in key is pressed, the motor 42 rotates forwards, and the laparoscope 1 moves out of the abdominal cavity; and when the zoom-out key is pressed, the motor 42 rotates reversely, and the laparoscope 1 moves into the abdominal cavity.

In yet another embodiment, referring to FIG. 9, the foot controller 7 is a separately provided structure, and can be connected to the direction controlling mechanism 3 and the moving mechanism 4 via data lines; alternatively, the foot controller 7 is wirelessly connected to the direction controlling mechanism 3 and the moving mechanism 4. Specifically, the foot controller 7 can transmit control signals to the direction controlling mechanism 3 and the moving mechanism 4 by means of Bluetooth, or data lines.

Specifically, the foot controller 7 further includes a trackball, wherein the trackball is connected to the up key, the down key, the left key and the right key. The trackball rolls leftwards to control the left key for action, the trackball rolls rightwards to control the right key for action, the trackball rolls upwards to control the up key for action, and the trackball rolls downwards to control the down key for action. The control of the trackball herein belongs to the prior art, which can refer to the principle of the trackball mouse, which will not be described in detail herein. In this way, the up, down, left, right offsets of the laparoscope 1 can be controlled by feet, the zoom-in key is pressed by the toe, and the zoom-out key is pressed by the heel, so that it is more convenient to control the laparoscope 1.

The foregoing embodiments are merely intended to illustrate the technical solutions of the present disclosure, but not to limit the present disclosure. Although the present disclosure is described in detail with reference to the foregoing embodiments, persons of ordinary skill in the art should understand that they may still make modifications to the technical solutions described in the foregoing embodiments, or make equivalent replacements to some or all technical features thereof; these modifications or replacements do not make the essence of the corresponding technical solutions depart from the scope of the technical solutions of the embodiments of the present disclosure, and shall all belong to the scope of the claims and description of the present disclosure.

1-13. (canceled)

14. A single-operator laparoscope auxiliary operating device, comprising:
a laparoscope;
universal rotating mechanisms, wherein two universal rotating mechanisms are provided on the laparoscope, each of the universal rotating mechanisms comprises a ball sleeve and a sleeve plate, the ball sleeve is slidably sleeved on the laparoscope, the sleeve plate is sleeved in a middle of the ball sleeve, and the ball sleeve is universally rotatable on the sleeve plate;
a direction controlling mechanism for controlling the sleeve plate away from a lens of the laparoscope to move in a plane;
a moving mechanism for controlling the laparoscope to slide in the ball sleeve;
a supporting mechanism for supporting the direction controlling mechanism;
a connecting frame, one end of the connecting frame is fixedly connected to the sleeve plate close to the lens of the laparoscope, and another end of the connecting frame is fixedly connected to the supporting mechanism; and
a foot controller connected to the direction controlling mechanism and the moving mechanism, for controlling operations of the direction controlling mechanism and the moving mechanism.

15. The single-operator laparoscope auxiliary operating device according to claim 14, wherein the direction controlling mechanism comprises a rectangular rail, four sliding sleeves, a first telescopic member, a second telescopic member, a third telescopic member and a fourth telescopic member;
the rectangular rail is fixedly mounted on the supporting mechanism, and the rectangular rail is formed by four rail edges;
the four sliding sleeves are slidably mounted on each of the rail edges of the rectangular rail respectively; and
the first telescopic member, the second telescopic member, the third telescopic member and the fourth telescopic member are in correspondence with the four sliding sleeves one by one; one end of each of the first telescopic member, the second telescopic member, the third telescopic member and the fourth telescopic member is fixedly connected to a corresponding sliding sleeve, and another end of each of the first telescopic member, the second telescopic member, the third telescopic member and the fourth telescopic member is fixedly connected to the sleeve plate away from the lens of the laparoscope.

16. The single-operator laparoscope auxiliary operating device according to claim 14, wherein the moving mechanism comprises a fixed sleeve, a motor, a threaded rod and a pushing structure;
the fixed sleeve is sleeved on one end of the laparoscope away from the lens of the laparoscope, and the fixed sleeve is fixedly connected to one end of the ball sleeve away from the lens of the laparoscope;
the motor is mounted in the fixed sleeve, the motor drives the threaded rod to rotate, and the foot controller controls the motor to rotate forwards and reversely;
the pushing structure is slidably connected in the fixed sleeve, the pushing structure is detachably connected to the laparoscope, and the pushing structure is in threaded connection with the threaded rod.

17. The single-operator laparoscope auxiliary operating device according to claim 14, wherein the supporting mechanism comprises a base, a height adjusting member, a length adjusting member and a fixing frame;
the base is fixedly connected to one end of the height adjusting member;
another end of the height adjusting member is vertically and fixedly connected to one end of the length adjusting member;
another end of the length adjusting member is hinged to the fixing frame;
the fixing frame is fixedly connected to the direction controlling mechanism, and the fixing frame is fixedly connected to one end of the connecting frame away from the sleeve plate.

18. The single-operator laparoscope auxiliary operating device according to claim 17, wherein universal wheels are mounted at a bottom of the base.

19. The single-operator laparoscope auxiliary operating device according to claim 14, further comprising a self-locking mechanism, the self-locking mechanism is mounted on the ball sleeve close to the lens of the laparoscope, and when the direction controlling mechanism operates, the self-locking mechanism locks the laparoscope and the ball sleeve close to the lens of the laparoscope; when the moving mechanism operates, the self-locking mechanism releases the laparoscope and the ball sleeve close to the lens of the laparoscope.

20. The single-operator laparoscope auxiliary operating device according to claim 19, wherein the self-locking mechanism is provided with a pan-tilt.

21. The single-operator laparoscope auxiliary operating device according to claim 16, wherein the pushing structure comprises a moving block, a friction column, a reset spring, a pushing rod, a rotating plate, a cam, and a limiting rod;
the moving block is slidably connected in the fixed sleeve, and the moving block is in threaded connection with the threaded rod;
the friction column is located in the moving block and abuts against the threaded rod;
one end of the reset spring is fixedly connected to the friction column, and another end of the reset spring is fixedly connected to the rotating plate;
the pushing rod is slidably provided in the moving block one end of the pushing rod is provided with a rotating groove, and the rotating plate is rotatably connected in the rotating groove; another end of the pushing rod is detachably connected to the laparoscope;
the cam is located in the rotating groove and is coaxially fixed on the rotating plate;
one end of the limiting rod is fitted with the cam, and another end of the limiting rod is slidably inserted through the rotating groove and is frictionally connected to the moving block.

22. The single-operator laparoscope auxiliary operating device according to claim 14, further comprising a protrusion edge and limiting mechanisms, each of the limiting mechanisms comprises arc rods and spiral springs;
one end of the ball sleeve is provided with the protrusion edge;
the sleeve plate is provided with arc groove, one end of each of the arc rods is slidably arranged in a corresponding arc groove, another end of each of the arc rods abuts against a corresponding protrusion edge, and the arc rods are concentrically arranged with the ball sleeve;
each of the spiral springs is provided on a corresponding sleeve plate, a central end of each of the spiral springs is fixedly connected to the sleeve plate, and an outer end of each of the spiral springs is fixedly connected to a corresponding arc rod; each of the spiral springs is able to drive the corresponding arc rod to reset.

23. The single-operator laparoscope auxiliary operating device according to claim 22, wherein one end of each of the arc rods abutting against the corresponding protrusion edge is provided with a rolling ball.

24. The single-operator laparoscope auxiliary operating device according to claim 14, wherein the foot controller is provided with an up key, a down key, a left key, a right key, a zoom-in key and a zoom-out key;
the up key and the down key are both connected to the first telescopic member and the second telescopic member;
the left key and the right key are both connected to the third telescopic member and the fourth telescopic member;
the zoom-in key and the zoom-out key are connected to the motor.

25. The single-operator laparoscope auxiliary operating device according to claim 24, wherein the foot controller is detachably mounted on the supporting mechanism.

26. The single-operator laparoscope auxiliary operating device according to claim 25, wherein the foot controller is connected to the direction controlling mechanism and the moving mechanism via data lines; alternatively, the foot controller is wirelessly connected to the direction controlling mechanism and the moving mechanism.

27. The single-operator laparoscope auxiliary operating device according to claim 15, wherein the foot controller is provided with an up key, a down key, a left key, a right key, a zoom-in key and a zoom-out key;
the up key and the down key are both connected to the first telescopic member and the second telescopic member;
the left key and the right key are both connected to the third telescopic member and the fourth telescopic member;
the zoom-in key and the zoom-out key are connected to the motor.

28. The single-operator laparoscope auxiliary operating device according to claim 16, wherein the foot controller is provided with an up key, a down key, a left key, a right key, a zoom-in key and a zoom-out key;
the up key and the down key are both connected to the first telescopic member and the second telescopic member;
the left key and the right key are both connected to the third telescopic member and the fourth telescopic member;
the zoom-in key and the zoom-out key are connected to the motor.
